(19) United States
(12) Patent Application Publication
(10) Pub. No.: US 2003/0036693 A1
(43) Pub. Date: Feb. 20, 2003

(54) Method to obtain the cardiac gating signal using a cardiac displacement sensor

(76) Inventors: Gopal B. Avinash, New Berlin, WI (US); Cherik Bulkes, Sussex, WI (US)

Correspondence Address: Patrick S. Yoder, Fletcher, Yoder & Van Someren, P.O. Box 692289, Houston, TX, 77269-2289, US

(21) Appl. No.: 09/928,029

(22) Filed: Aug. 10, 2001

(51) Int. Cl. A61B 5/05

(52) U.S. Cl. 600/413

(57) ABSTRACT

A technique is disclosed for predicting a future occurrence of an activity for data acquisition timing. The disclosed technique can predict mechanical activity, such as physiological activity, to facilitate acquisition timing for a data acquisition system, which may comprise an imaging assembly, a physiological diagnostic assembly, or other acquisition assemblies.

### FIELD OF THE INVENTION

The present invention relates generally to the field of imaging systems, such as those used for medical diagnostic applications. More particularly, the invention relates to a technique for controlling an imaging system to acquire images at a desired state in a physiological cycle, such as a motionless state, to enhance image quality

### BACKGROUND OF THE INVENTION

In many applications, it is often desirable to obtain an image at a particular point in a variable cycle, such as a peak of the variable cycle, to analyze behavior at that peak. In the medical field, imaging systems are often used to obtain internal physiological information of a subject. For example, a medical imaging system may be used to obtain images of the bone structure, the brain, the heart, the lungs, and various other features of a subject Medical imaging systems include magnetic resonance imaging (MRI) systems, computed tomography (CT) systems, x-ray systems, ultrasound systems, and various other imaging modalities.

By way of example, magnetic resonance imaging (MRI) systems have become ubiquitous in the field of medical diagnostics. Over the two past decades, improved techniques for MRI examinations have been developed that now permit very high-quality images to be produced in a relatively short time. As a result, diagnostic images with varying degrees of resolution are available to the radiologist that can be adapted to particular diagnostic applications. For example, MRI systems are used for the diagnostic evaluation of the aorta and peripheral vascular system. MRI systems also can provide images of the left ventricular and the right ventricular, including measurements of ejection fraction, heart wall motion, and perfusion.

In general, MRI examinations are based on the interactions among a primary magnetic field, a radiofrequency (RF) magnetic field and time varying magnetic gradient fields with nuclear spins within the subject of interest. Specific nuclear components, such as hydrogen nuclei in water molecules, have characteristic behaviors in response to external magnetic fields. The precession of spins of such nuclear components can be influenced by manipulation of the fields to obtain RF signals that can be detected, processed, and used to reconstruct a useful image.

The magnetic fields used to produce images in MRI systems include a highly uniform, static magnetic field that is produced by a primary magnet. A series of gradient fields are produced by a set of three gradient coils disposed around the subject. The gradient fields encode positions of individual volume elements or voxels in three dimensions. A radiofrequency coil is employed to produce an RF magnetic field. This RF magnetic field perturbs the spin system from its equilibrium direction, causing the spins to precess around the axis of their equilibrium magnetization. During this precession, radiofrequency fields are emitted by the spins and detected by either the same transmitting RF coil, or by a separate receive-only coil. These signals are amplified, filtered, and digitized. The digitized signals are then processed using one of several possible reconstruction algorithms to reconstruct a useful image.

Many specific techniques have been developed to acquire MR images for a variety of applications. One major difference among these techniques is in the way gradient pulses and RF pulses are used to manipulate the spin systems to yield different image contrasts, signal-to-noise ratios, and resolutions. Graphically, such techniques are illustrated as “pulse sequences” in which the pulses are represented along with temporal relationships among them. In recent years, pulse sequences have been developed which permit extremely rapid acquisition of a large amount of raw data. Such pulse sequences permit significant reduction in the time required to perform the examinations. Time reductions are particularly important for acquiring high resolution images, as well as for suppressing motion effects and reducing the discomfort of patients in the examination process.

In MRI systems, as with many other medical imaging systems, images are often desired in physiological features undergoing cyclical movement. Unfortunately, the cyclical movement causes motion artifacts in the image. To minimize motion artifacts, an image acquisition sequence may be gated to the physiological cycle (e.g., a cardiac cycle, a respiratory cycle, etc.). However, the physiological cycle can vary over time. This complicates the gating process. In cardiac imaging, an electrocardiogram (ECG) may be used to measure electroactivity before motion occurs and, thereby, facilitate image acquisition at the desired state of the cardiac. However, there are many disadvantages of using convention ECG techniques for image acquisition control. For example, conventional techniques do not measure the actual physiological activity to control the image acquisition timing. Moreover, electrocardiograms require actual “intrusive” contact with the subject that may interfere with some diagnostic systems. Conventional techniques also fail to provide accurate timing control that can keep up in real-time with the time varying physiological motions, such as cardiac activity.

There is a need, therefore, for an improved technique of triggering image acquisition. In particular, a technique is needed for predicting physiological activity, and specific events thereof, based on actual physiological activity. There is also a need for a real-time correction technique suitable for adjusting timing predictions based on prior occurrences of actual physiological activity.

### SUMMARY OF THE INVENTION

The present invention provides a technique for predicting a future occurrence of an activity for data acquisition timing. The disclosed technique can predict mechanical activity, such as physiological activity, to facilitate acquisition timing for a data acquisition system, which may comprise an imaging assembly, a physiological diagnostic assembly, or other acquisition assemblies.

An aspect of the present technique provides a method of triggering an imaging system. The method includes sensing physiological activity, isolating an event in the physiological activity, and predicting a future occurrence of the event for triggering an imaging system.

Another aspect of the present technique provides a timing system for a diagnostic system. The system includes a processing assembly adapted for signal processing and prediction, wherein the processing assembly includes a port for a sensor, a signal separator, an interval estimator, and an event predictor. The port is adapted to receive an activity signal from the sensor. The signal separator is adapted to isolate at least one cyclical pattern from the activity signal. The interval estimator is adapted to estimate a time interval between successive cycles of the at least one cyclical pattern. The event predictor is adapted to predict a desired state of the at least one cyclical pattern for a diagnostic system.

### BRIEF DESCRIPTION OF THE DRAWINGS

FIG. 1 is a diagrammatical representation of an imaging system for use in medical diagnostic imaging and implementing certain aspects of the present technique;

FIG. 2 is a block diagram of functional components of an exemplary pulse sequence description module in a controller for a system of the type illustrated in FIG. 1;

FIG. 3 is a graphical representation of an exemplary pulse sequence description for an MRI examination that may be implemented in the system of FIG. 1;

FIG. 4 is flow chart illustrating exemplary features of the present technique with reference to the imaging system of FIG. 1 and signal charts of FIGS. 5-8;

FIG. 5 is a signal chart of mechanical activity according to certain aspects of the present technique;

FIG. 6 is a signal chart of desired mechanical activity isolated from the mechanical activity of FIG. 5;

FIG. 7 is a signal chart of electrical activity corresponding to the desired mechanical activity of FIG. 6; and

FIG. 8 is a combined signal chart of the desired mechanical activity and the electrical activity of FIGS. 6 and 7.

### DETAILED DESCRIPTION OF THE INVENTION

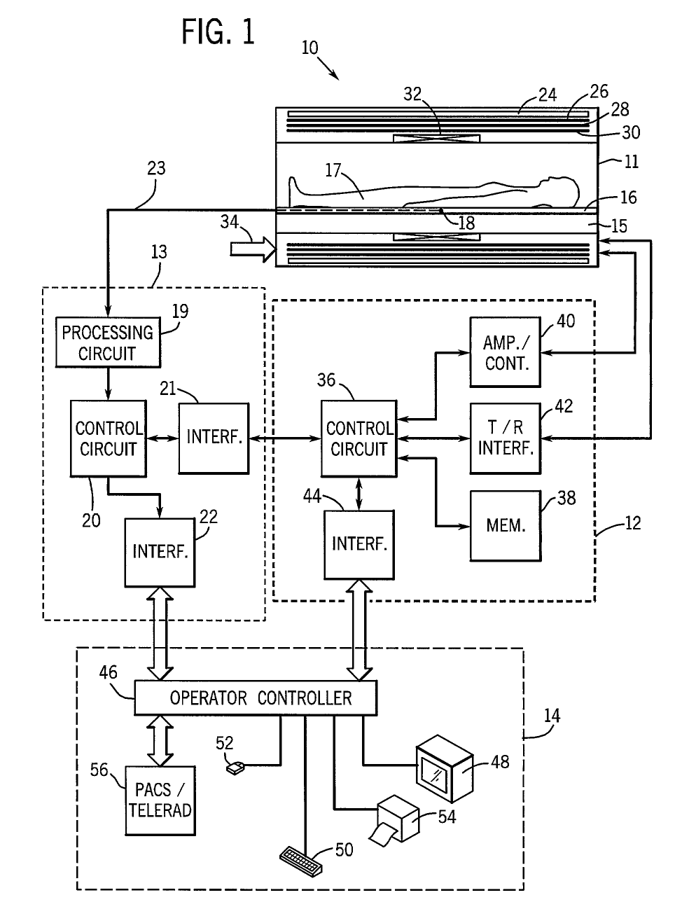

Turning now to the drawings, FIG. 1 illustrates an exemplary embodiment of the present technique. The following discussion illustrates the present technique in context of medical modalities and, specifically, in context of an imaging system 10. It should be noted that the unique aspects of the present technique are also beneficial and suitable for many other systems and applications. For example, the imaging system 10 may comprise one or more independent or integrated medical diagnostic systems, such as a magnetic resonance imaging (MRI) system, a computed tomography (CT) imaging system, an x-ray system, an ultrasound system, and other systems suitable for desired medical modalities.

In FIG. 1, the imaging system 10 is illustrated as a magnetic resonance imaging system having a scanner 11, scanner control circuitry 12, coordination circuitry 13, and system control circuitry 14. Although the imaging system 10 may comprise any suitable scanner or detector, the imaging system 10 is conveniently illustrated as a full body scanner having a patient bore 15 into which a table 16 can be positioned to place a patient 17 in a desired position for scanning. Scanner 11 can be of any suitable type of rating, including scanners varying from 0.5 Tesla ratings to 1.5 Tesla ratings and beyond.

As discussed in greater detail below, the coordination circuitry 13 facilitates monitoring of sensed physiological parameters and coordination with the scanner control circuitry 12 for the scanner 11. The coordination circuitry 13 illustrated in FIG. 1 includes a sensor assembly 18, a processing circuit 19, a control circuit 20, a communication interface 21, and an interface 22. The sensor assembly 18 is communicatively coupled to the coordination circuitry via a communication assembly 23, which may be an analog or digital cable assembly, a wireless communication system, or other suitable communication systems. The sensor assembly 18 monitors desired physiological activity of the patient 17 and transmits data to the processing circuit 19 for storage, analysis, characterization, and various other processing. As discussed below, the processing circuit 19 generates a predicted time for a future physiological event based on the monitored physiological activity and transmits the prediction to the control circuit 20 for coordination of the predicted time with the scanner control circuitry 12 and the scanner 11. Accordingly, the control circuit 20 communicates the predicted time to a control circuit 36 of the scanner control circuitry 12 via the communication interface 21. In operation, the predicted time facilitates accurate timing for data acquisition by the scanner 11 to obtain data (e.g., image data) at desired phases of physiological mechanical activity (e.g., respiration, heart, etc.). The control circuit 20 is also coupled to the system control circuitry 14 via the interface 22, which facilitates monitoring, processing, maintenance, calibration and other control of the coordination circuitry 13.

The coordination circuitry 13, and the components 18-23, may include a variety of hardware and software suitable for the desired acquisition system. For example, the processing circuit 19 may include one or more processors, circuit boards, storage/memory devices (e.g., tape drive, disk drive, optical drive, hard drive, RAM, ROM, cache, etc.), power supplies, communication devices and ports, input/output devices and ports (e.g., keyboard, monitor, mouse, printer, etc.), and any other desired components. Moreover, the processing circuit 19 may include various software applications, such as custom and standard operating systems, data analysis applications, databases, graphical applications, statistical analysis applications, sensor monitoring and analysis applications, and prediction routines for predicting future events from past activities. The software applications may be stored locally or remotely, provided that the coordination circuitry can access and utilize the applications.

The control circuit 20, the communication interface 21, and the interface 22 also may include hardware and software, such as that described above, to perform their respective functions. For example, the control circuit 20 may include a circuit board, a processor, and memory for interpreting a predicted time from the processing circuit and for adapting the predicted time to the desired acquisition system, such as the imaging system 10. The communication interface 21 may include a network or communication board, a processor, and memory to facilitate transformation of the predicted time information to a format acceptable and interpretable by the control circuit 36 of the scanner control circuitry 12. The interface 22 also may have a communication board, a processor, and memory adapted to facilitate communication and interaction with the system control circuitry 14. The components 19-22 may be configured to have independent memory, processors, circuit boards and software, or the coordination circuitry 13 may embody an integrated assembly having a central processor, memory, circuits and software.

The sensor assembly 18 may include one or more motion activity sensors, an electrocardiogram (ECG) sensor, and various other physiological sensors. As illustrated, the sensor assembly 18 includes a non-intrusive mechanical/motion sensor disposed in the table 16 adjacent the patient 17. However, the sensor assembly 18 may embody an array of sensors disposed throughout the table 16 or throughout the interior of the scanner 11. To monitor cardiac or respiratory activity, the illustrated orientation of the sensor assembly 18 may facilitate accurate monitoring of internal mechanical activity of the patient 17. In other diagnostic systems, such as those specialized for a particular physiological analysis, the sensor assembly 18 may be oriented in any suitable position to ensure accurate reading without interfering with the diagnostic system. The sensor assembly 18 can also have one or more sensors disposed on the patient 17, either intrusively or non-intrusively. While a single sensor may be sufficient for the present technique, the use of multiple sensors can enhance accuracy of the analysis performed by the coordination circuitry 13. For example, multiple types of sensors may be used collaboratively to improve measurements of a desired parameter (e.g., heart) or to facilitate isolation of the desired parameter by measuring different physiological activities and screening out undesirable activity signals. In operation, the processing circuit 19 can utilize the sensed parameters and known physiological activity patterns to predict behavior of the desired physiological parameter for enhanced data acquisition timing of the imaging system 10.

In the illustrated embodiment, a non-intrusive type of the sensor assembly 18 is particularly well-suited for use with the image system 10. For example, a passive device for sensing sound, vibration, or other parameters associated with mechanical movement can be used to monitor physiological activity. An active device also can be used to measure mechanical activity. For example, mechanical activity can be monitored by directing a pulse of energy, sound, vibration, or other pulses into a medium (e.g., the patient 17) and then calculating activity based on its reflection timing, amplitude, and properties of the medium. Statistical methods also may be used to improve the accuracy of such techniques. Although a variety of sensor types and technologies can be utilized within the scope of the present technique, a suitable sensor technology is produced by C-All Technologies, Ashqelon, Israel.

As discussed above, the coordination circuitry 13 is suitable for a broad range of data and image acquisition systems, including medical diagnostic and imaging systems as illustrated in FIG. 1. The imaging system 10, which is illustrated as an MRI system, may include a variety of standard, optional, and custom components for operation with or without the coordination circuitry 13. Moreover, the imaging system 10 may be networked together via analog, digital and/or wireless cables, and may have one or more systems or components networked at a remote location. For example, the imaging system 10 may have the scanner control circuitry 12, the coordination circuitry 13 and/or the system control circuitry 14 networked to the scanner 11 from a separate room, a separate building, or an entirely separate geographic location or business entity. Also, the imaging system 10 may provide network or Internet access (e.g., secure access) to facilitate interaction between physicians, service technicians, or others to ensure accurate diagnosis of a patient.

Although various scanner types and configurations can be employed in the imaging system 10 (e.g., MRI system), the scanner 11 illustrated in FIG. 1 includes a series of associated coils for producing controlled magnetic fields, for generating radiofrequency excitation pulses, and for detecting emissions from gyromagnetic material within the patient in response to such pulses. In the illustrated embodiment of imaging system 10, a primary magnet coil 24 is provided for generating a primary magnetic field generally aligned with patient bore 15. A series of gradient coils 26, 28 and 30 are grouped in a coil assembly for generating controlled magnetic gradient fields during examination sequences as described more fully below. A radiofrequency coil 32 is provided for generating radiofrequency pulses for exciting the gyromagnetic material. As illustrated in FIG. 1, coil 32 can also serve as a receiving coil. Thus, RF coil 32 may be coupled with driving and receiving circuitry in passive and active modes for receiving emissions from the gyromagnetic material and for applying radiofrequency excitation pulses, respectively. Alternatively, various configurations of receiving coils may be provided separate from RF coil 32. Such coils may include structures specifically adapted for target anatomies, such as head coil assemblies, and so forth. Moreover, receiving coils may be provided in any suitable physical configuration, including phased array coils, and so forth. The present technique also may include a radio frequency shield positioned between the gradient coils (e.g., gradient coils 28 and 30) to shield the RF magnetic field from the gradient coils, which may be affected by the field during operation.

In a present configuration, the gradient coils 26, 28 and 30 have different physical configurations adapted to their function in the imaging system 10. As will be appreciated by those skilled in the art, the coils are comprised of conductive wires, bars or plates which are wound or cut to form a coil structure which generates a gradient field upon application of control pulses as described below. The placement of the coils within the gradient coil assembly may be done in several different orders, but in the present embodiment, a Z-axis coil is positioned at an innermost location, and is formed generally as a solenoid-like structure, which has relatively little impact on the RF magnetic field. Thus, in the illustrated embodiment, gradient coil 30 is the Z-axis solenoid coil, while coils 26 and 28 are Y-axis and X-axis coils respectively.

The coils of scanner 11 are controlled by external circuitry to generate desired fields and pulses, and to read signals from the gyromagnetic material in a controlled manner. As will be appreciated by those skilled in the art, when the material, typically bound in tissues of the patient, is subjected to the primary field, individual magnetic moments of the paramagnetic nuclei in the tissue partially align with the field. While a net magnetic moment is produced in the direction of the polarizing field, the randomly oriented components of the moment in a perpendicular plane generally cancel one another. During an examination sequence, an RF frequency pulse is generated at or near the Larmor frequency of the material of interest, resulting in rotation of the net aligned moment to produce a net transverse magnetic moment. This transverse magnetic moment precesses around the main magnetic field direction, emitting RF signals that are detected by the scanner and processed for reconstruction of the desired image.

Gradient coils 26, 28 and 30 serve to generate precisely controlled magnetic fields, the strength of which vary over a predefined field of view, typically with positive and negative polarity. When each coil is energized with known electric current, the resulting magnetic field gradient is superimposed over the primary field and produces a desirably linear variation in the Z-axis component of the magnetic field strength across the field of view. The field varies linearly in one direction, but is homogenous in the other two. The three coils have mutually orthogonal axes for the direction of their variation, enabling a linear field gradient to be imposed in an arbitrary direction with an appropriate combination of the three gradient coils.

The pulsed gradient fields perform various functions integral to the imaging process. Some of these functions are slice selection, frequency encoding and phase encoding. These functions can be applied along the X-, Y- and Z-axis of the original coordinate system or along other axes determined by combinations of pulsed currents applied to the individual field coils.

The slice select gradient determines a slab of tissue or anatomy to be imaged in the patient. The slice select gradient field may be applied simultaneously with a frequency selective RF pulse to excite a known volume of spins within a desired slice that precess at the same frequency. The slice thickness is determined by the bandwidth of the RF pulse and the gradient strength across the field of view.

The frequency encoding gradient is also known as the readout gradient, and is usually applied in a direction perpendicular to the slice select gradient. In general, the frequency-encoding gradient is applied before and during the formation of the MR echo signal resulting from the RF excitation. Spins of the gyromagnetic material under the influence of this gradient are frequency encoded according to their spatial position along the gradient field. By Fourier transformation, acquired signals may be analyzed to identify their location in the selected slice by virtue of the frequency encoding.

Finally, the phase encode gradient is generally applied before the readout gradient and after the slice select gradient. Localization of spins in the gyromagnetic material in the phase encode direction is accomplished by sequentially inducing variations in phase of the precessing protons of the material using slightly different gradient amplitudes that are sequentially applied during the data acquisition sequence. The phase encode gradient permits phase differences to be created among the spins of the material in accordance with their position in the phase encode direction.

As will be appreciated by those skilled in the art, a great number of variations may be devised for pulse sequences employing the exemplary gradient pulse functions described above as well as other gradient pulse functions not explicitly described here. Moreover, adaptations in the pulse sequences may be made to appropriately orient both the selected slice and the frequency and phase encoding to excite the desired material and to acquire resulting MR signals for processing.

The coils of scanner 11 are controlled by scanner control circuitry 12 to generate the desired magnetic field and radiofrequency pulses. In the diagrammatical view of FIG. 1, control circuitry 12 thus includes a control circuit 36 for commanding the pulse sequences employed during the examinations, and for processing received signals. Control circuit 36 may include any suitable programmable logic device, such as a CPU or digital signal processor of a general purpose or application-specific computer. Control circuit 36 further includes memory circuitry 38, such as volatile and nonvolatile memory devices for storing physical and logical axis configuration parameters, examination pulse sequence descriptions, acquired image data, programming routines, and so forth, used during the examination sequences implemented by the scanner.

Interface between the control circuit 36 and the coils of scanner 11 is managed by amplification and control circuitry 40 and by transmission and receive interface circuitry 42. Circuitry 40 includes amplifiers for each gradient field coil to supply drive current to the field coils in response to control signals from control circuit 36. Interface circuitry 42 includes additional amplification circuitry for driving RF coil 32. Moreover, where the RF coil serves both to emit the radiofrequency excitation pulses and to receive MR signals, circuitry 42 will typically include a switching device for toggling the RF coil between active or transmitting mode, and passive or receiving mode. A power supply, denoted generally by reference numeral 34 in FIG. 1, is provided for energizing the primary magnet 24. Finally, circuitry 12 includes interface components 44 for exchanging configuration and image data with system control circuitry 14. It should be noted that, while in the present description reference is made to a horizontal cylindrical bore imaging system employing a superconducting primary field magnet assembly, the present technique may be applied to various other configurations, such as scanners employing vertical fields generated by superconducting magnets, permanent magnets, electromagnets or combinations of these means.

System control circuitry 14 may include a wide range of devices for facilitating interface between an operator or radiologist and scanner 11 via scanner control circuitry 12. In the illustrated embodiment, for example, an operator controller 46 is provided in the form of a computer work station employing a general purpose or application-specific computer. The station also typically includes memory circuitry for storing examination pulse sequence descriptions, examination protocols, user and patient data, image data, both raw and processed, and so forth. The station may further include various interface and peripheral drivers for receiving and exchanging data with local and remote devices. In the illustrated embodiment, such devices include a conventional computer keyboard 50 and an alternative input device such as a mouse 52. A printer 54 is provided for generating hard copy output of documents and images reconstructed from the acquired data. A computer monitor 48 is provided for facilitating operator interface. In addition, system 10 may include various local and remote image access and examination control devices, represented generally by reference numeral 56 in FIG. 1. Such devices may include picture archiving and communication systems, teleradiology systems, and the like.

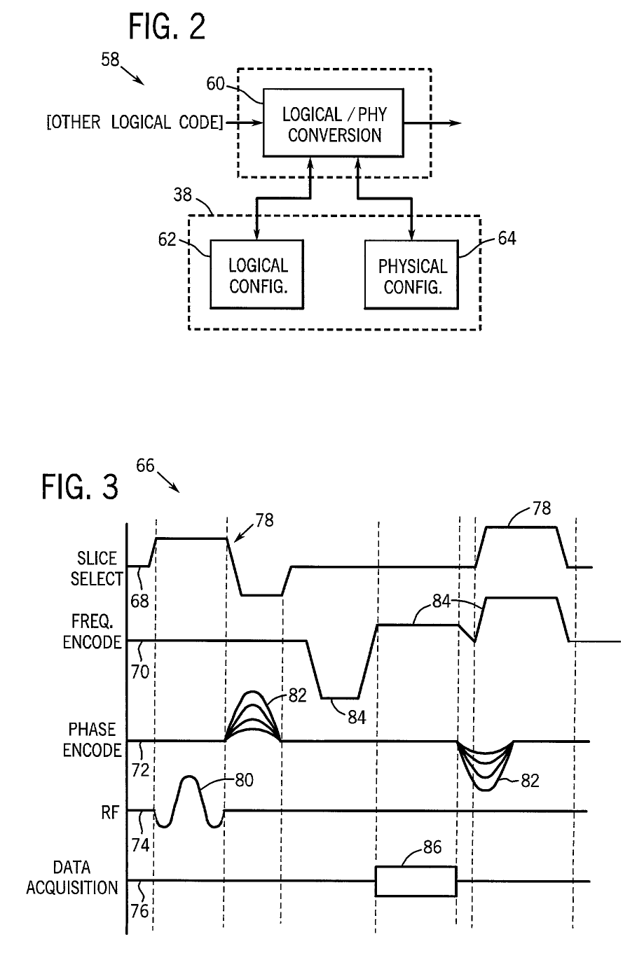

In general, the pulse sequences implemented in the MRI system will be defined both by functional and physical configuration sets and parameter settings stored within control circuitry 12. FIG. 2 diagrammatically represents relationships between functional components of control circuit 36 and configuration components stored with memory circuitry 38. The functional components facilitate coordination of the pulse sequences to accommodate preestablished settings for both functional and physical axes of the system. In general, the axis control modules, denoted collectively by reference numeral 58, include a functional-to-physical module 60 which is typically implemented via software routines executed by control circuit 36. In particular, the conversion module is implemented through control routines that define particular pulse sequences in accordance with preestablished imaging protocols.

When called upon, code defining the conversion module references functional sets 62 and physical configuration sets 64. The functional configuration sets may include parameters such as pulse amplitudes, beginning times, time delays, and so forth, for the various logical axes described above. The physical configuration sets, on the other hand, will typically include parameters related to the physical constraints of the scanner itself, including maximum and minimum allowable currents, switching times, amplification, scaling, and so forth. Conversion module 60 serves to generate the pulse sequence for driving the coils of scanner 11 in accordance with constraints defined in these configuration sets. The conversion module will also serve to define adapted pulses for each physical axis to properly orient (e.g. rotate) slices and to encode gyromagnetic material in accordance with desired rotation or reorientations of the physical axes of the image.

By way of example, FIG. 3 illustrates a typical pulse sequence that may be implemented on a system such as that illustrated in FIG. 1 and calling upon configuration and conversion components such as those shown in FIG. 2. While many different pulse sequence definitions may be implemented, depending upon the examination type, in the example of FIG. 3, a gradient recalled acquisition in steady state mode (GRASS) pulse sequence is defined by a series of pulses and gradients appropriately timed with respect to one another. The pulse sequence, indicated generally by reference numeral 66, is thus defined by pulses on a slice select axis 68, a frequency-encoding axis 70, a phase encoding axis 72, an RF axis 74, and a data acquisition axis 76. In general, the pulse sequence description begins with a pair of gradient pulses on slice select axis 68 as represented at reference numeral 78. During a first of these gradient pulses, an RF pulse 80 is generated to excite gyromagnetic material in the subject. Phase encoding pulses 82 are then generated, followed by a frequency encoding gradient 84. A data acquisition window 86 provides for sensing signals resulting from the excitation pulses which are phase and frequency encoded. The pulse sequence description terminates with additional gradient pulses on the slice select, frequency encoding, and phase encoding axes.

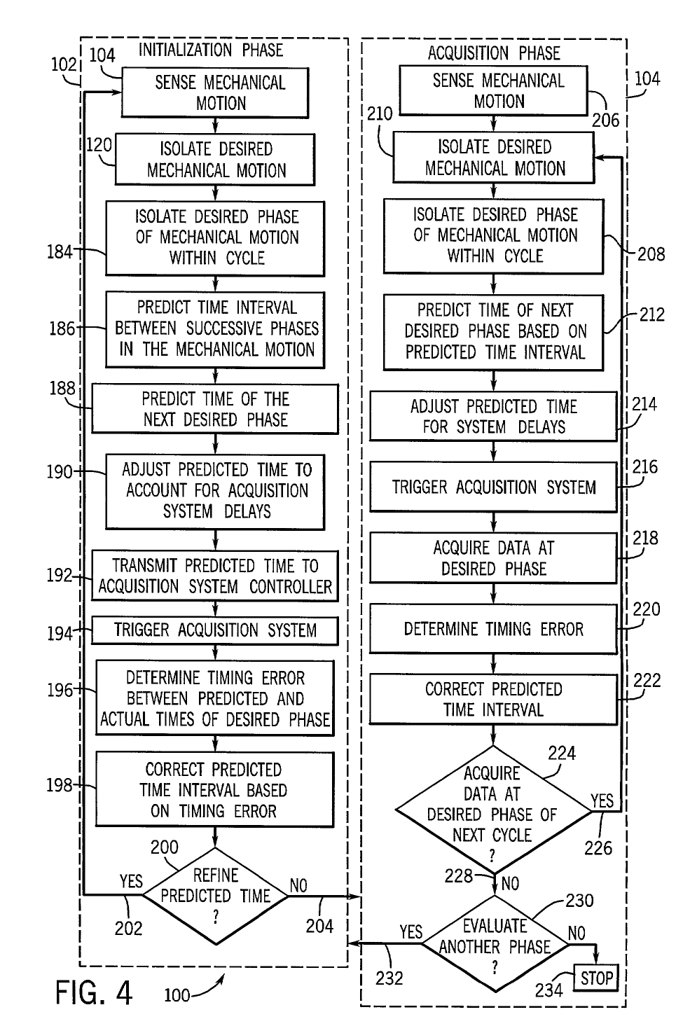

FIG. 4 is a flow chart of the present technique illustrating an exemplary coordination process 100, which can be used in conjunction with data acquisition systems. As illustrated, the coordination process 100 includes an initialization phase 102 and an acquisition phase 104 associated with timing prediction and control for various applications, such as imaging, medical diagnosis, and other desirable applications and data acquisition systems that may benefit from timing control. Accordingly, the coordination process 100 will be discussed in context of the imaging system 10, and particularly the coordination circuitry 13, of FIG. 1 to illustrate unique aspects of the present technique.

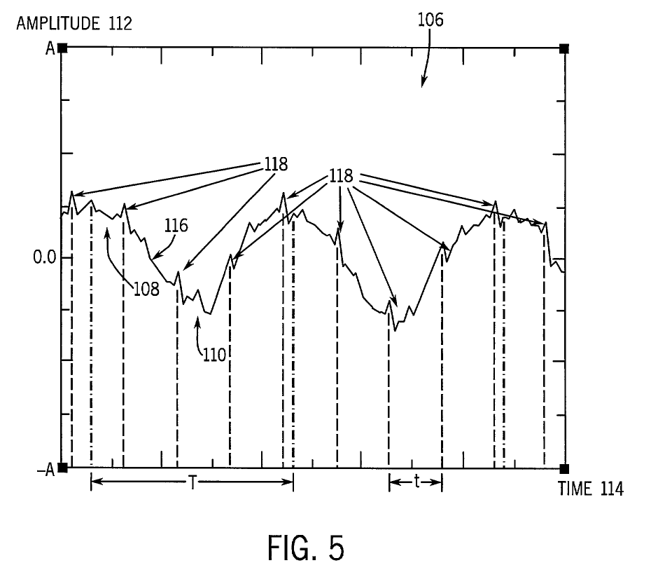

In the initialization phase 102, the coordination process 100 senses mechanical motion (block 104), such as illustrated in FIG. 5, via a processing system such as the sensor assembly 18 and the coordination circuitry 13 illustrated in FIG. 1. The mechanical motion, or mechanical activity, may be associated with one or a plurality of mechanical activities, which may be independent or related to one another in some way. Thus, the mechanical motion sensed (block 104) by the initialization phase 102 can provide a cumulative signal representing multiple activities sensed by the sensor assembly 18. For example, FIG. 5 is a signal chart of an exemplary signal pattern 106 corresponding to the mechanical motion sensed (block 104) by the sensor assembly 18 for analysis and processing by the coordination circuitry 13. As illustrated, the signal pattern 106 cycles between amplitudes 108 and 110 on an amplitude axis 112 (e.g., actual measurement units or normalized) over time along a time axis 114. In this sensed mechanical motion (block 104), the signal pattern 106 may represent a plurality of superimposed signals corresponding to the sensed mechanical motion (block 104), which corresponds to physiological activities of a subject. For example, the signal pattern 106 has an activity signal 116 corresponding to respiratory activity (e.g., breathing), an activity signal 118 corresponding to cardiovascular activity (e.g., cardiac), and a plurality of other sensed mechanical motions (block 104). In the illustrated signal pattern 106, the activity signal 116 has an interval and a frequency of approximately T and 1T, respectively, for one of its cycles. In comparison, the activity signal 118 has a relatively smaller amplitude and higher frequency than the activity signal 116. The activity signal 118 has an interval and a frequency of approximately t and 1/t, respectively, for one of its cycles. However, for both activity signals 116 and 118, the intervals, amplitudes and frequencies may vary over time. This time-varying nature of the activity signals complicates the analysis and processing of the underlying mechanical motions, such as the sensed mechanical motions (block 104).

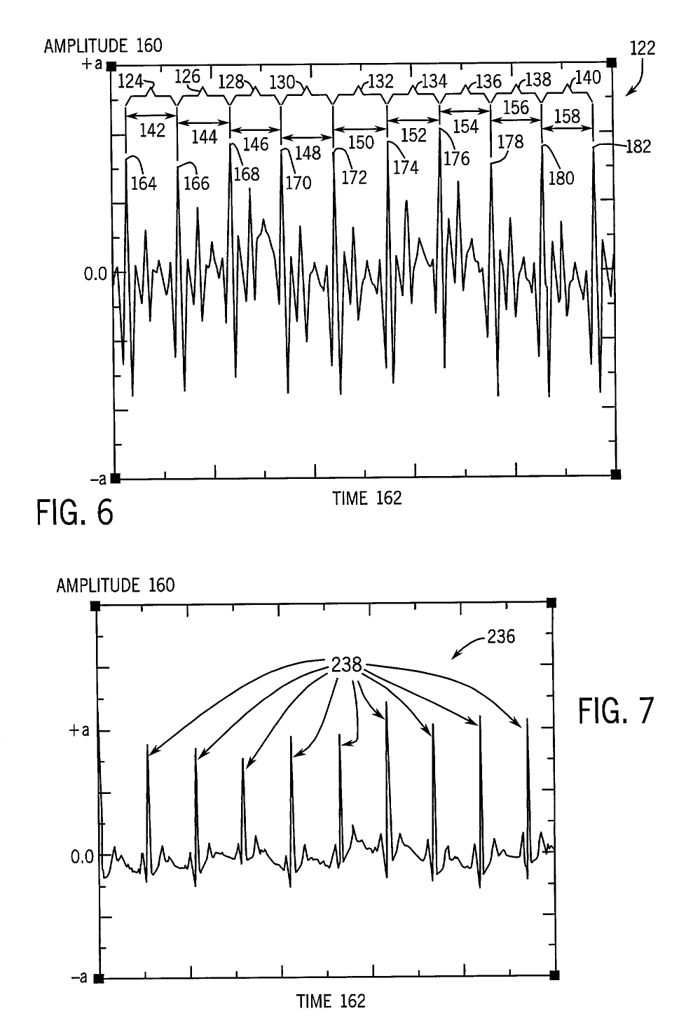

The initialization phase 102 of the coordination process 100 proceeds to isolate desired mechanical motion (block 120) from the mechanical motion sensed (block 104) by the sensor assembly 18. For example, the processing circuit 19 may filter, separate, transform and process the sensed mechanical motion (block 104) to obtain an isolated signal 122, as illustrated in FIG. 6. However, if the signal pattern 106 corresponds to the desired activity (e.g., a single or independent mechanical activity), then the act of isolating the desired mechanical motion (block 120) may simply include refinement and clarification of the signal pattern 106 to obtain the isolated signal 122.

In the illustrated signals of FIGS. 5 and 6, the isolated signal 122 may correspond to the activity signal 118, which may represent cardiac displacement of a live subject. If the signal pattern 106 includes a plurality of independent or distinguishable signals, then a variety of techniques can be used to separate the desired signal. One or more activity signals may be distinguishable and separable according to known characteristics, such as frequency ranges, amplitude ranges, and interrelationships with other signals and parameters. For example, known physiological parameters, such as cardiovascular and respiratory parameters, can be used to facilitate isolation of a desired activity such as cardiac activity. As discussed above, breathing patterns are characterized by relatively low frequency, high amplitude signals relative to cardiac patterns. The processing circuit 19 can utilize these differing frequencies and amplitudes to facilitate signal separation and isolation. Moreover, the present technique may employ multiple sensors to monitor one or more activities related to the desired mechanical motion (block 120). For example, the sensor assembly 18, or separate sensors, can be used to independently monitor pulse rates, breathing rates, electrical activity, and other characteristics. This may be done prior to operation of the present technique, or concurrent with the present technique, to provide a reference for isolating the desired mechanical motion (block 120). Thus, the desired mechanical motion (block 120) can be isolated from the sense mechanical motion (block 104) based on known activity characteristics (e.g., known physiological characteristics, known cyclical patterns, etc.) and sensed parameters.

In FIG. 6, the isolated signal 122 corresponds to the activity signal 118 (e.g., cardiac activity) isolated from the signal pattern 106, which may have been sensed by the sensor assembly 18. As illustrated, the isolated signal 122 has cycles 124, 126, 128, 130, 132, 134, 136, 138 and 140 successively occurring over time in time intervals 142, 144, 146, 148, 150, 152, 154, 156 and 158, respectively. The isolated signal 122 has a cyclical pattern, which cycles between positive and negative amplitudes (e.g., +a and −a) on an amplitude axis 160 (e.g., actual measurement units or normalized) over a time axis 162. The isolated signal 122 also may have patterns within each cycle and trends over a series of cycles, which can vary over time. For example, over a series of successive cycles, the isolated signal 122 may exhibit a change in amplitude, a change in time interval, or a positive or negative shift of the entire signal relative to the amplitude axis 160 or the time axis 162. In the isolated signal 122 illustrated in FIG. 6, the changes in amplitude correspond to various events occurring in the underlying mechanical activity, such as cardiovascular, respiratory, or other physiological activity. For example, the isolated signal 122 has a plurality of peaks, such as peaks 164, 166, 168, 170, 172, 174, 176, 178, 180 and 182, which may correspond to ventricular contraction or other events of the cardiac a cycle.

In accordance with the coordination process 100 illustrated in FIG. 4, the initialization phase 102 analyzes the isolated signal 122 and isolates a desired phase of mechanical motion within the cycle (block 184). For example, the coordination circuitry 13 may be used to isolate the peaks 164-182 or other phases in the underlying mechanical activity to facilitate accurate timing for data acquisition in the acquisition phase 104. In the illustrated isolated signal 122, the peaks 164-182 relate to ventricular contraction of a cardiac. The peaks 164-182, or another desired phase in the cycle, may be isolated with a phase identification module. For example, the processing circuit 19 may include a peak signal identification module, which utilizes a dynamically varying threshold that searches for a maximum signal over a predetermined interval. The phase identification module may be a software application or appropriate circuitry of the processing circuit 19. After identifying the desired phase in the cycles, the coordination process 100 predicts time intervals between successive phases in the mechanical motion (block 186). For example, the previous time intervals may be determined by isolating the peaks 164-182 and by measuring peak-to-peak distances. The time intervals also may be analyzed to determine trends, expectations, average values, and other relevant characteristics of the prior cycles 124-140. This time interval analysis facilitates a prediction of the future behavior of the underlying mechanical motion related to the isolated signal 122.

Accordingly, future mechanical activity and specific occurrences of events can be predicted using a variety of statistical or data analysis techniques incorporated in the coordination circuitry 13. For example, one technique calculates an expected valve of the time difference between successive phases in the mechanical activity (block 186). The expected time difference may be defined as the average value of time intervals for a specified number of prior cycles. The time intervals can be measured between successive peaks, such as peaks 164-182, or between other desired phases of mechanical activity. Thus, the expected time interval may be defined as:

$$dt = {\sum }_{n = 1}^{N} \frac{Δ {t}_{n}}{N}$$

where Δtₙ is the time interval for cycle n of the N total cycles being evaluated to obtain the expected time interval dt. If the isolated signal 122 is associated with cardiac events, then the predicted time interval (block 186) may represent the expected time interval dt between successive occurrences of cardiac activity, such as ventricular contractions. Accordingly, the coordination process 100 predicts the time of the next desired phase (block 188) based on the predicted time interval (block 186). For example, the predicted time of a future occurrence of the desired phase can be defined as:

T_{P}=T_{R}+dt

where T_{R} is the time of the previous occurrence of the desired phase and dt is the estimated or predicted time interval. Thus, the present technique predicts a future activity based on prior actual mechanical activity.

Once the initialization phase 102 has predicted the time interval (block 186) and the future occurrence of the desired activity (block 188), the initialization phase 102 adjusts the time prediction (e.g., T_{P}) to account for system delays (block 190). For example, the imaging system 10 may have various delays in its subcomponents, such as the scanner 11, the scanner control assembly 12, the coordination circuitry 13, and the system control circuitry 14. The system delays can be attributed to conduction delays in the sensor assembly 18 and coordination circuitry 13 during activity sensing, or the system delays may be attributed to conduction delays associated with triggering the scanner 11. Accordingly, the predicted time T_{P} is adjusted (block 190) to provide a triggering signal more accurately representing the time at which the scanner 11 must be triggered to acquire data corresponding to the desired activity. The triggering signal, or control signal T_{C}, may be defined as an adjusted time prediction:

T_{C}=T_{P}−α*dt

where α is a timing adjustment factor ranging between 0<α<1 to account for system delays, which may be attributed to a variety of factors. The timing adjustment factor α can be determined from the relative relationship between left ventricular contraction and the mitral valve opening in a cardiac signal, from empirical analysis, or from other suitable techniques.

The control signal T_{C} is utilized by the imaging system 10 to facilitate accurate timing and data acquisition by the scanner 11. Accordingly, the coordination process 100 transmits the predicted time (e.g., the control signal T_{C}) to the acquisition system (block 192) and the acquisition system is triggered (block 194) to obtain data at the desired phase. In the initialization phase 102, the data may or may not be acquired, but the accuracy of the control signal T_{C} is evaluated to determine if there is any timing error between the time prediction and the actual time for the desired phase (block 196). The prediction error E_{P} can be computed as:

E_{P}=T_{A}−T_{P}

where T_{A} is the actual time that the desired phase occurred. Using the prediction error E_{P}, the coordination process 100 corrects the predicted time interval dt to account for the new time interval (block 198) and to facilitate real time correction and refinement of the time predictions. Thus, the expected time interval dt may be recalculated as:

$$dt = {dt}^{′} + {E}_{P} = \frac{dt * N + Δ {t}^{′}}{N + 1} + {E}_{P}$$

where Δt′ is the actual time interval for the predicted cycle and N+1 is the total cycles being evaluated to obtain the updated expected time interval dt′. Accordingly, the expected time interval dt facilitates a more accurate prediction, and real-time correction, of the time interval between the previously predicted phase and the next desired phase. Although the initialization phase 102 may be utilized a single time to obtain the expected time interval dt, which is used to obtain the control signal T_{C}, the initialization phase 102 may be repeated to refine the predicted time interval dt, the predicted time T_{P}, and the control signal T_{C}. Thus, the initialization phase 102 may provide an option to refine the time predictions (block 200). For example, the processing circuit 19 may request user input, or it may automatically repeat the initialization phase 102 to refine the expected time interval dt if the prediction error exceeds a specified threshold. If refinement is desired, then the initialization phase 102 returns to block 104 for another time prediction analysis 202. The initialization phase may repeat one or more of the blocks 104, 120, 184 and 186, but may skip directly to block 188 for prediction of the next desired phase. Otherwise, the initialization phase 102 continues to the acquisition phase 104 to operate and control the acquisition system 204.

As illustrated in FIG. 4, the acquisition phase 104 utilizes the expected time interval dt provided by the initialization phase 102 to facilitate accurate timing for triggering the acquisition system (e.g., the imaging system 10). This phase is very similar to the initialization phase 102 except that it readily utilizes the previously determined expected time interval dt between the desired phases (e.g., cardiac peak signals). Accordingly, the acquisition phase 104 senses mechanical motion (block 206) and isolates a desired mechanical motion (block 208) from the sensed motion (block 206), as illustrated in FIGS. 5 and 6, respectively. The acquisition phase 104 then isolates the desired phase of activity within each cycle of the desired mechanical motion (block 210), as discussed above.

Using the expected time interval dt provided by the initialization phase 102, the acquisition phase 104 predicts the time of a future occurrence of the desired phase (block 212). As discussed above, the predicted time T_{P} can be defined as:

T_{P}=T_{R}+dt

where T_{R} is the time of the previous occurrence of the desired phase and dt is the estimated or predicted time interval. The predicted time T_{P} is then adjusted to account for system delays (block 214). As discussed above, the triggering signal, or control signal T_{C}, may be defined as an adjusted time prediction:

T_{C}=T_{P}−α*dt

where α is a timing adjustment factor ranging between 0<α<1 to account for system delays.

Utilizing the control signal T_{C}, the acquisition phase 104 triggers the acquisition system (block 216) to obtain data at the desired phase (block 218). Accordingly, the expected time interval dt, determined from prior occurrences of the desired phase, facilitates accurately timed acquisition by the imaging system 10. For example, the acquisition system may acquire a particularly well-timed and accurate visualization of the desired phase, which may be a cardiac event in the case of cardiac imaging. The result is a phase-locked data set, or image, corresponding to the desired phase. Moreover, the timing is based on actual mechanical motion rather than electrical activity (e.g., ECG), thereby providing direct correlation between activity and data acquired by the acquisition system. Thus, the present technique enhances characterization and analysis of healthy physiological motion, identification of abnormalities, and general medical diagnosis and treatment.

As in the initialization phase 102, the acquisition phase 104 determines the timing error E_{P} between the actual time T_{A} and the predicted time T_{P} for the desired phase (block 220) and corrects the expected time interval dt (block 222). Thus, the prediction error E_{P} may be calculated from:

E_{P}=T_{A}−T_{P}

The prediction error E_{P} is then used to correct the predicted time interval dt to account for the new time interval (block 222). The updated time interval prediction can be calculated as follows:

$$dt = {dt}^{′} + {E}_{P} = \frac{dt * N + Δ {t}^{′}}{N + 1} + {E}_{P}$$

where Δt′ is the actual time interval for the predicted cycle and N+1 is the total cycles being evaluated to obtain the updated expected time dt′. Accordingly, the expected time interval dt provides a more accurate prediction, and real-time correction, of the time interval between the previously predicted phase and the next desired phase.

Although the acquisition phase 104 may simply acquire data for a single occurrence of the desired phase, the coordination process 100 can provide an option to acquire additional data at the desired phase of the next cycle (block 224). Accordingly, if the user or acquisition system commands further analysis of the desired phase 226, then the coordination process 100 may return to block 206 for another sequence of the acquisition phase 104. However, one or more of the blocks 206, 208 and 210 may be skipped or simply compared with the previous performance of the respective blocks. If further analysis of the desired phase is not undertaken, then the acquisition phase 104 may provide an option to evaluate another phase of mechanical activity (block 230). Accordingly, if a new analysis is desired, then the coordination process 100 returns to the initialization phase 102 for a new analysis and time interval prediction 232. If no further analysis is desired, then the coordination process 100 may simply stop (block 234).

It is important to point out that the present technique is particularly well suited for phase-locked data acquisition of cyclical events, such as cardiac events or other physiological events in a living subject. The system and methods disclosed herein can be used to predict mechanical motion based on prior mechanical motion, prior knowledge, and other sensed parameters. Moreover, the present technique may enhance the accuracy of predictions based on continuous refinement and correction in real-time. Thus, the present technique differs substantially from conventional techniques, which use electrocardiograms (ECG's) to control data acquisition triggering of imaging systems. In contrast to the mechanical motions sensed by the sensor assembly 18, electrocardiograms relate to electrical activity associated with the mechanical activity. The present technique may sense electrical activity, and then predict and correct a timing signal based on subsequent actual activity. Moreover, the present technique may benefit from simultaneous use of both electrocardiograms and mechanical activity monitoring to improve prediction and correction.

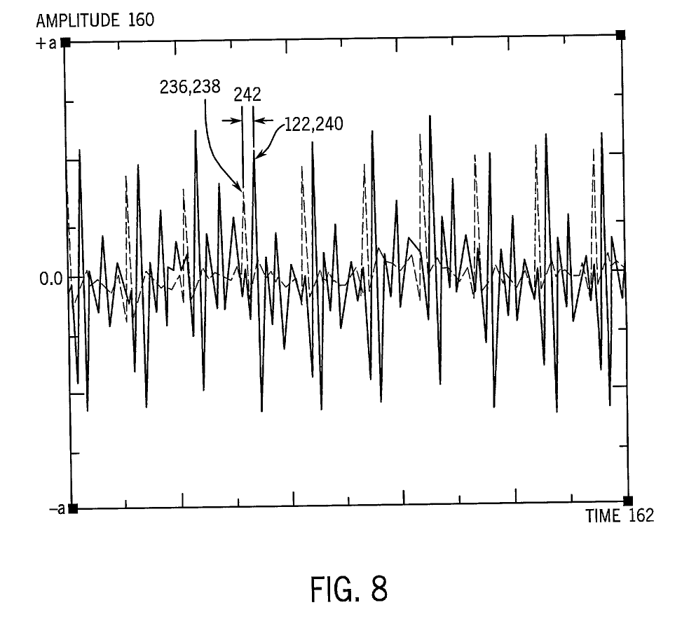

FIG. 7 is a signal chart illustrating an electrocardiogram signal 236 over an identical time frame as the isolated signal 122 illustrated in FIG. 6. The signals are drastically different. FIG. 8 is a combined signal chart having both the electrocardiogram of FIG. 7 and the isolated signal 122 (e.g., mechanical activity signal) of FIG. 6 over the identical time frame. As illustrated, FIGS. 6-8 include the amplitude axis 160 and the time axis 162 to facilitate comparison. The amplitude axis 160 also may be normalized to facilitate comparison between the isolated signal 122 and the electrocardiogram signal 236. The electrocardiogram signal 236 may be used to analyze the cardiac, but it measures electrical activity rather than actual mechanical movement. Electrical activity generally occurs before the mechanical activity, as illustrated by the time lag between the electrocardiogram signal 236 and the isolated signal 122 of mechanical activity. Although the peaks 238 in the electrocardiogram correspond to electrical activity for a cardiac event (e.g., ventricular contraction), the actual cardiac event 240 does not occur until a time period 242 lapses after the electrical activity. Accordingly, the present technique uses prior activity (e.g., electrical, mechanical, etc.) to predict future occurrences of a mechanical activity, and may correct its prediction in real-time after each actual occurrence of mechanical activity.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A method of triggering an imaging system, comprising:
sensing physiological activity;
isolating an event in the physiological activity; and
predicting a future occurrence of the event for triggering an imaging system.

2. The method of claim 1, wherein sensing physiological activity comprises mechanically sensing internal physiological activity.

3. The method of claim 1, wherein sensing physiological activity comprises non-intrusively sensing internal physiological activity.

4. The method of claim 1, wherein sensing physiological activity comprises sensing motion of an internal organ of a subject.

5. The method of claim 1, wherein sensing physiological activity comprises sensing a plurality of physiological parameters.

6. The method of claim 1, wherein sensing physiological activity comprises sensing internal mechanical activity of a subject.

7. The method of claim 6, wherein sensing internal mechanical activity comprises sensing cardiovascular activity of the subject.

8. The method of claim 7, wherein sensing cardiovascular activity comprises sensing cardiac activity.

9. The method of claim 6, wherein sensing internal mechanical activity comprises sensing respiratory activity of the subject.

10. The method of claim 9, wherein sensing respiratory activity comprises sensing lung activity.

11. The method of claim 1, wherein isolating the event comprises analyzing the physiological activity over a time interval.

12. The method of claim 1, wherein isolating the event comprises isolating a desired activity from the physiological activity.

13. The method of claim 12, wherein isolating the event comprises identifying cyclical patterns in the physiological activity.

14. The method of claim 12, wherein isolating the event comprises separating the desired activity based on known motion characteristics of the desired activity.

15. The method of claim 13, wherein isolating the event comprises filtering at least a portion of the cyclical patterns having frequencies outside of an expected frequency range for the desired activity.

16. The method of claim 12, wherein isolating the event comprises identifying a desired phase in a cycle of the desired activity.

17. The method of claim 16, wherein identifying the desired phase comprises identifying a peak amplitude in the cycle.

18. The method of claim 1, wherein isolating the event comprises isolating a repeating point in a cyclical signal corresponding to an internal organ of a subject.

19. The method of claim 18, wherein isolating the event comprises isolating a cardiovascular event of the subject.

20. The method of claim 18, wherein isolating the event comprises isolating a respiratory event of the subject.

21. The method of claim 1, wherein predicting the future occurrence comprises analyzing historical behavior of the physiological activity.

21. The method of claim 1, wherein analyzing historical behavior comprises calculating an expected time interval between successive occurrences of the event.

22. The method of claim 21, wherein predicting the future occurrence comprises determining a reference time based on a previous occurrence of the event and adding the expected time interval to provide a predicted time for the future event.

23. The method of claim 1, wherein predicting the future occurrence comprises adjusting a predicted time to account for system response delays in the imaging system.

24. The method of claim 1, comprising controlling timing of an image acquisition component of the imaging system.

25. The method of claim 1, comprising acquiring a desired image of the event.

26. The method of claim 25, wherein acquiring the desired image of the event comprises obtaining image data of a cardiac phase.

27. The method of claim 1, comprising calculating a prediction error between a predicted time and an actual time of the future occurrence.

28. The method of claim 27, comprising adjusting the predicted time based on the prediction error.

29. The method of claim 27, wherein adjusting the predicted time comprises adjusting a predicted time interval between successive occurrences of the event based on the prediction error.

30. A method of medical diagnosis, comprising:
analyzing internal mechanical activity of a subject;
predicting a cyclical event of the internal mechanical activity; and
facilitating acquisition of physiological data via a diagnostic system at a future time based on the cyclical event predicted.

31. The method of claim 30, wherein analyzing internal mechanical activity comprises sensing physiological activity.

32. The method of claim 31, wherein sensing physiological activity comprises non-intrusively sensing physiological motion.

33. The method of claim 31, wherein sensing physiological activity comprises sensing motion of an internal organ of a subject.

34. The method of claim 31, wherein sensing physiological activity comprises sensing activity of a plurality of physiological features.

35. The method of claim 31, wherein sensing physiological activity comprises sensing cardiovascular activity of the subject.

36. The method of claim 30, wherein analyzing internal mechanical activity comprises isolating a desired activity from the internal mechanical activity.

37. The method of claim 36, wherein isolating the desired activity comprises identifying activity patterns in the internal mechanical activity.

38. The method of claim 37, wherein isolating the desired activity comprises dividing the activity patterns based at least partially on known activity characteristics.

39. The method of claim 36, wherein isolating the desired activity comprises obtaining a cyclical signal having distinguishable characteristics.

40. The method of claim 36, wherein isolating the desired activity comprises identifying a recurring physiological event.

41. The method of claim 30, wherein analyzing internal mechanical activity comprises temporally identifying a relatively motionless phase of a cyclical physiological motion.

42. The method of claim 30, wherein predicting the cyclical event comprises temporally estimating a future occurrence of a physiological event.

43. The method of claim 42, wherein temporally estimating the future occurrence comprises estimating a future cardiovascular event.

44. The method of claim 42, wherein temporally estimating the future occurrence comprises estimating a future respiratory event.

45. The method of claim 30, wherein predicting the cyclical event comprises calculating an expected time interval between successive cycles of the internal mechanical activity.

46. The method of claim 30, wherein facilitating acquisition of physiological data comprises adjusting a time prediction of the cyclical event to account for system response delays.

47. The method of claim 30, comprising providing a triggering signal adapted to trigger a data acquisition unit based at least partially on the cyclical event predicted.

48. The method of claim 30, comprising acquiring physiological data of the internal mechanical activity at the future time.

49. The method of claim 48, wherein acquiring physiological data comprises acquiring data representative of a desired image.

50. The method of claim 49, wherein acquiring data representative of the desired image comprising acquiring data representative of a cardiovascular event.

51. The method of claim 30, comprising acquiring image data of the cyclical event at the future time.

52. The method of claim 30, comprising calculating a prediction error between an actual time and a predicted time of the cyclical event predicted.

53. The method of claim 52, comprising adjusting the predicted time based on the prediction error.

54. A phase-locking system for a physiological diagnostic system, comprising:
a sensor assembly adapted to sense mechanical physiological activity;
a processor assembly coupled to the sensor assembly and adapted to predict physiological activity based at least partially on mechanical physiological activity sensed by the sensor assembly; and
a control assembly coupled to the processor assembly and adapted to generate a control signal for a physiological diagnostic system based on the physiological activity predicted by the processor assembly.

55. The phase-locking system of claim 54, wherein the sensor assembly comprises a non-intrusive sensor.

56. The phase-locking system of claim 54, wherein the sensor assembly comprises a plurality of motion sensors.

57. The phase-locking system of claim 54, wherein the sensor assembly comprises a sensor adapted to sense respiratory activity.

58. The phase-locking system of claim 54, wherein the sensor assembly comprises a sensor adapted to sense cardiovascular activity.

59. The phase-locking system of claim 54, wherein the sensor assembly comprises a sensor adapted to sense a plurality of physiological features of a subject.

60. The phase-locking system of claim 54, wherein the processor assembly comprises a filter for separating at least one signal corresponding to an independent activity of the mechanical physiological activity.

61. The phase-locking system of claim 54, wherein the processor assembly comprises a signal analysis module adapted to evaluate cyclical patterns of the mechanical physiological activity.

62. The phase-locking system of claim 61, wherein the signal analysis module comprises an interval analyzer adapted to estimate a time interval between successive cycles of the mechanical physiological activity.

63. The phase-locking system of claim 54, wherein the processor assembly comprises an event prediction module adapted to calculate a predicted time for a desired phase of the mechanical physiological activity.

64. The phase-locking system of claim 63, wherein the event prediction module comprises a system configuration module adapted to adjust the predicted time based on system response delays.

65. The phase-locking system of claim 63, wherein the event prediction module comprises a prediction correction module adapted to adjust the predicted time based on differences between an actual time and the predicted time for the desired phase.

66. The phase-locking system of claim 54, wherein control assembly comprises a communication system adapted to interface with physiological diagnostic system.

67. The phase-locking system of claim 54, wherein communication system is adapted to interface with a medical imaging system.

68. The phase-locking system of claim 54, comprising a medical diagnostic system coupled to the control assembly and adapted to acquire physiological data.

69. The phase-locking system of claim 68, wherein the medical diagnostic system comprises an imaging unit.

70. The phase-locking system of claim 69, wherein imaging unit comprises a magnetic resonance imaging unit.

71. An imaging system, comprising:
an image acquisition device;
control circuitry coupled to the image acquisition device;
a motion sensor oriented to sense activity affecting a targeted image region of the image acquisition device; and
processor circuitry coupled to the motion sensor and adapted to analyze and predict the activity for acquisition timing of the image acquisition device.

72. The imaging system of claim 71, wherein the image acquisition device comprises a medical imaging assembly.

73. The imaging system of claim 72, wherein the medical imaging assembly comprises a magnetic resonance imaging system.

74. The imaging system of claim 71, wherein the control circuitry comprises an acquisition-timing module.

75. The imaging system of claim 71, wherein the motion sensor comprises a non-intrusive sensor assembly adapted to sense mechanical activity.

76. The imaging system of claim 75, wherein the mechanical activity comprises physiological activity of a subject.

77. The imaging system of claim 76, wherein physiological activity comprises cardiovascular activity.

78. The imaging system of claim 71, wherein the processor circuitry comprises a signal analysis module adapted to estimate time intervals between successive cycles of a cyclic activity.

79. The imaging system of claim 78, wherein the signal analysis module comprises a prediction module adapted to calculate a predicted time for a future occurrence of a desired event of the cyclical activity.

80. The imaging system of claim 71, comprising a communication interface between the processor circuitry and the control circuitry.

81. A timing system for a diagnostic system, comprising:
a processing assembly adapted for signal processing and prediction, the processing assembly comprising:
a port adapted to receive an activity signal from a sensor;
a signal separator adapted to isolate at least one cyclical pattern from the activity signal;
an interval estimator adapted to estimate a time interval between successive cycles of the at least one cyclical pattern; and
an event predictor adapted to predict a desired state of the at least one cyclical pattern for a diagnostic system.

82. The timing system of claim 81, comprising the diagnostic system coupled to the processing assembly.

83. The timing system of claim 81, wherein the diagnostic system comprises a medical diagnostic system.

84. The timing system of claim 81, wherein the diagnostic system comprises a physiological imaging system.

85. The timing system of claim 81, comprising the sensor, wherein the sensor comprises a mechanical activity sensor.

86. The timing system of claim 85, wherein the sensor comprises a physiological activity sensor.

87. The timing system of claim 85, wherein the sensor comprises a cardiac activity sensor.

88. The timing system of claim 81, wherein processing assembly comprises an acquisition-timing trigger adapted to trigger the diagnostic system at a predicted time for the desired state.

89. The timing system of claim 81, wherein the processing assembly comprises a system configuration module adapted to adjust a predicted time for the desired state based on system response delays.

90. The timing system of claim 81, wherein the processing assembly comprises a prediction correction module adapted to adjust a predicted time for the desired state based on differences between an actual time and the predicted time for the desired state.
